(19) United States
(12) Patent Application Publication
(10) Pub. No.: US 2020/0211700 A1
(43) Pub. Date: Jul. 2, 2020

(54) PERFORMANCE OPPORTUNITY ANALYSIS SYSTEM AND METHOD

(71) Applicant: 3M INNOVATIVE PROPERTIES COMPANY, St. Paul, MN (US)

(72) Inventor: Jeremy M. Zasowski, Stoneham, MA (US)

(21) Appl. No.: 16/724,529

(22) Filed: Dec. 23, 2019

Related U.S. Application Data
(60) Provisional application No. 62/786,646, filed on Dec. 31, 2018.

(51) Int. Cl. G16H 40/20; G16H 10/60; G16H 50/70

(52) U.S. Cl. CPC G16H 40/20; G16H 50/70; G16H 10/60

(57) ABSTRACT

A computer implemented method includes obtaining access to a database containing patient healthcare data that includes multiple performance measures, analyzing the healthcare data to identify sets of patients sharing one or more of the identifications, analyzing the identified sets of patients to identify a group from one of the identified sets, the group including patients having a primary performance measure worse than a performance benchmark associated with the diagnosis category and severity of illness level of the one of the identified sets, analyzing secondary performance measures of the identified group to identify secondary performance measures correlated to the primary performance measure being worse than the performance benchmark, and analyzing the healthcare data to identify associated details of patient care in the group corresponding to the primary performance measure being worse than the performance benchmark.

### CROSS REFERENCE TO RELATED APPLICATION

This application claims the benefit of U.S. Provisional Patent Application No. 62/786,646, filed Dec. 31, 2018, which is incorporated herein by reference in its entirety.

### BACKGROUND

Efficiency of service delivered by a healthcare provider is an important consideration for various stakeholders, including patient, providers, and other organizations. A health care provider generally refers to any provider of health care services and can encompass a broad range of entities, such as physician and/or non-physician health care practitioners, physician groups, facilities, health systems and accountable care organizations for example. Determination of the efficiency of healthcare providers and opportunities for improvement can be difficult to determine or identify. While there are many tools available using different approaches to identify quality of care, such tools are limited to the improvement of quality of care.

One prior attempt at determining efficiency creates data models and extracts data related to all patient encounters by a particular facility or for a particular service provided. The data includes patient information, outcome information, cost information, projected revenue, and other information related to cost and services performed. The extracted data is organized according to the models and stored for querying. Pre-defined queries are available to help identify areas where efficiency may be improved.

### SUMMARY

A computer implemented method includes obtaining access to a database containing patient healthcare data that includes multiple performance measures, analyzing the healthcare data to identify sets of patients sharing one or more of the identifications, analyzing the identified sets of patients to identify a group from one of the identified sets, the group including patients having a primary performance measure worse than a performance benchmark associated with the diagnosis category and severity of illness level of the one of the identified sets, analyzing secondary performance measures of the identified group to identify secondary performance measures correlated to the primary performance measure being worse than the performance benchmark, and analyzing the healthcare data to identify associated details of patient care in the group corresponding to the primary performance measure being worse than the performance benchmark.

### BRIEF DESCRIPTION OF THE DRAWINGS

FIG. 1 is a flowchart illustrating a computer implemented method 100 of identifying areas for improvement for various organization

FIG. 2 is a flowchart illustrating a method of analyzing the sets of patients to identify a group according to an example embodiment.

FIG. 3 is a graph illustrating the complexity of data associated with health care providers providing care to patients, represented as patient experience according to an example embodiment.

FIG. 4 is a graphical representation of a performance matrix that shows relations between different types of data according to an example embodiment.

FIG. 5 is a block diagram illustrating a method of generating the performance matrix according to an example embodiment.

FIG. 6 is a block flow diagram illustration operation of the system utilizing the performance matrix according to an example embodiment.

FIG. 7 is graphic representation of a performance improvement identification by the system according to an example embodiment.

FIG. 8 is a graphical representation of relevant causes for the primary performance measure of inpatient length of stay according to an example embodiment.

FIG. 9 is a graphical representation of relevant causes for the primary performance measure of potentially preventable hospital admissions according to an example embodiment.

FIG. 10 is a block schematic diagram of a computer system to implement one or more example embodiments.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using, software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

Prior methods that attempt to determine efficiency organize encounter data in a particular manner, store the data in a database, and then offer pre-determined or user created queries to generate reports indicative of efficiency. Such methods tend to lump all patient data into a database and fail to differentiate between patients.

In various embodiments of the inventive subject matter, a performance improvement evaluation system (PIES) includes an engine that groups of similar patients associated with poor performances on one or more performance measures are found. The groups may comprise clusters of patients that share commonality. Key contributors and causes related to the poor performance are identified, facilitating identification of areas for improvement in efficiency or performance of healthcare providers.

The combination of a group of patients with poor performance on one or more performance measures, along with the results of the findings as to the contributors and causes related to that poor performance is called “performance opportunity”.

In order to find performance opportunities, PIES analyzes healthcare data in order to find a patient group with poor performance on a performance measure when compared to a performance benchmark. The system then identifies the value of improving that patient group's performance from its current level, up to the associated benchmark level of performance. The net difference between the current performance level, and the benchmark level, is calculated to be the value of the opportunity.

Once the system finds a group of patients performing poorly on a specific performance measure (primary measure) the system then runs through a series of algorithms to identify the contributors and causes related to the poor performance. The groups may comprise clusters of patients that share commonality.

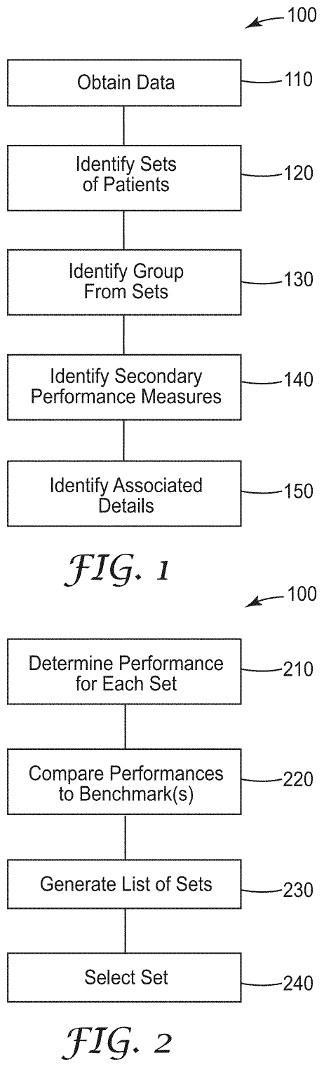

FIG. 1 is a flowchart illustrating a computer implemented method 100 of identifying areas for improvement for various organization. Method 100 may be applied in many different types of organizations. Examples primarily described are related to the provision of health care services by one or more healthcare facilities.

Method 100 begins at operation 110 by obtaining access to a database containing patient healthcare data that includes identifications of health care facility, service lines, diagnosis category, severity of illness level, and multiple performance measures.

At operation 120, the computer analyzes the healthcare data to identify sets of patients sharing one or more of the identifications.

The computer analyzes the identified sets of patients at operation 130 to identify a group from one of the identified sets. The group includes patients having a primary performance measure worse than a performance benchmark associated with the diagnosis category and severity of illness level of the one of the identified sets.

The primary performance measure in one embodiment is length of stay. Method 100 may determine a cost associated with an average difference between the length of stay and a benchmark length of stay, wherein the group is selected as the group having the highest cost. In further embodiments, the performance measures include two or more of length of stay, readmissions, complications, pharmacy, laboratory, emergency room visits, radiology, and mortality.

Analyzing the identified sets of patients at operation 130 to identify a group may include iteratively creating multiple sets as a function of at least one or more of health care facility, service lines, diagnosis category, and severity of illness. The primary measure or characteristic effectively draws the best circle around patients that best defines the group. This may be done by finding the strongest correlation between patients in a group. Many if not all permutations of groupings may be iteratively processed to find groups with high opportunity for cost savings. The highest contributing factors or causes for the group are identified. In some embodiments, the members of the group are included if they are above a threshold that is higher than a corresponding benchmark value. The thresholds may vary by client and may be adjusted to find the best opportunities for performance improvement.

At operation 140, the computer analyzes secondary performance measures of the identified group to identify secondary performance measures correlated to the primary performance measure being worse than the performance benchmark.

The computer at operation 150 analyzes the healthcare data to identify associated details of patient care in the group corresponding to the primary performance measure being worse than the performance benchmark. Such associated details are correlated to opportunities for performance improvements to increase efficiencies and reduce costs. Analyzing the healthcare data to identify the associated details is performed as a function of the identified secondary performance measures.

The associated details include one or more of attending physician, operating physician, patient age, patient zip code, nursing unit, admit day of the week, discharge day of the week, admit from location, and discharge to location. the associated details are ranked according to frequency of occurrence in the group to identify a focused area of potential improvement that is quantified by the associated difference.

FIG. 2 is a flowchart illustrating a method 200 of analyzing the sets of patients to identify a group. As mentioned above, operation 130 may include iteratively creating multiple sets in the process of identifying a group. Method 200 includes determining performance of each of the multiple sets at operation 210. At operation 220, the determined performance of each of the multiple sets is compared to corresponding performance benchmarks to determine a difference for each set. Operation 230 generates a list of sets based on the differences. Operation 240 selects the set with the largest difference as the identified group.

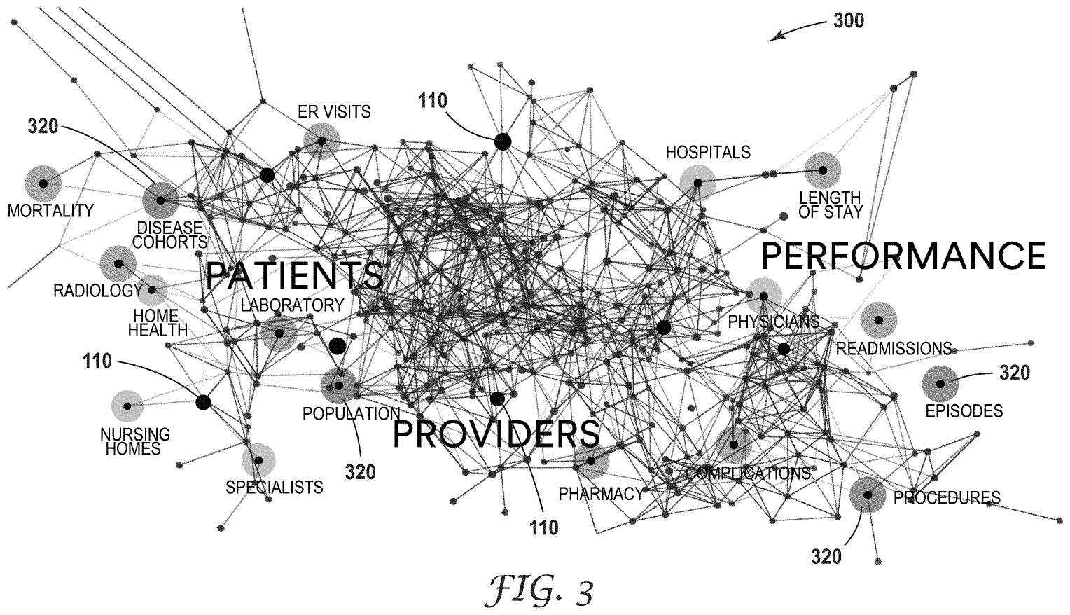

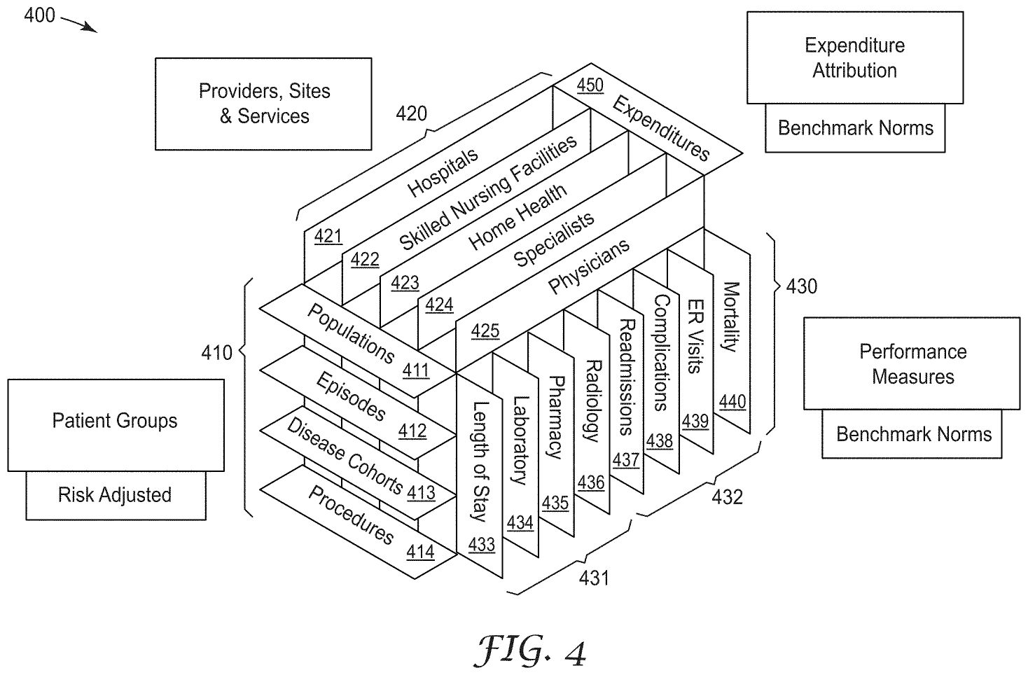

FIG. 3 is a graph 300 illustrating the complexity of data associated with health care providers providing care to patients, represented as patient experience. A number of providers represented at circles 310 and patients represented by circles 320 are illustrated. Example providers include hospitals, skilled nursing facilities, home health services, specialists, and physicians. Patient groups are illustrated as populations, episodes, disease cohorts, and procedures. As can be seen, interactions can vary significantly between patients and the providers that are involved in their care. The patients may have many different diagnoses and preexisting conditions. Thus, treating all patients the same in analysis of provider efficiencies can lead to data that is skewed by outliers. An outlier may be a patient with a preexisting condition that is much more likely to affect selected performance measures, such as length of stay of complications.

FIG. 4 is a graphical representation of a performance matrix 400 that shows relations between patient groups 410 that include planes representing populations 411, episodes 412, disease cohorts 413, and procedures 414 in a first dimension. Healthcare facilities, such as providers, sites, and services 420 are represented by planes intersecting the patient group 410 planes in a second dimension. Services 420 include hospitals 421, skilled nursing facilities 422, home health services 423, specialists 424, and physicians 425.

Performance measures 430 are represented by planes intersecting the other planes in a third dimension. Performance measures 430 may include two different types of performance measures, resources 431 and outcomes 432. Resources 430 are represented by length of stay 433, laboratory 434, pharmacy 435, and radiology 436. Outcomes 432 include readmissions 437, complications 438, emergency room (ER) visits 439, and mortality 440.

Note that each of sets of planes in the matrix 400 are shown as an example, and that more or fewer groups, services, and measures may be utilized in further embodiments. A further aspect of the performance matrix 400 include expenditure attribution 450. Such attribution ascribes a cost of each of the services rendered. Performance measures may include benchmark normal, which may be local to a particular facility or health care entity or may also be regional or national norms. Benchmarks may also be provided for best practices. The benchmarks may be based on historical data collected over periods of time from one or more healthcare providers.

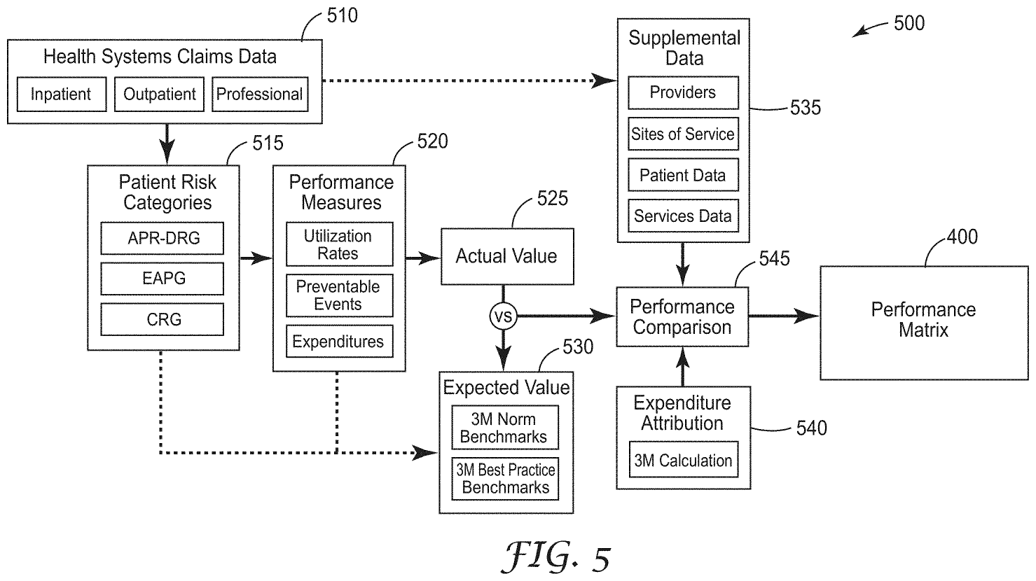

FIG. 5 is a block diagram illustrating a method 500 of generating the performance matrix 400. The performance matrix 400 includes data from multiple sources that is accumulated and processed. In one embodiment, data from a health system claims database 510 is obtained and organized into patient risk categories 515 and performance measures 520. The claims database 510 includes inpatient, outpatient, and professional services information regarding claims made based on services provided to patients. The patient risk categories 515 includes APR DRG (diagnostic related group) data, EAPG data, and CRG data. Actual values 525 are generated from the performance measures 520 data that is derived from the data in patient risk categories 515. The actual values may include utilization rates, preventable events, and expenditures for example, as well as other performance measures including those described above.

A data set of expected values 530 includes one or more benchmark values that correspond to the performance measure values. As mentioned above, many different benchmarks may be utilized. The actual values 525 may be used to augment the benchmark data.

Supplemental data 535 includes data from providers, sites of service, patient data, and services data that may not be included in the claims data 510. Expenditure attributions 540 provides information that attirbutes expenses to the entities that incurred the expenses. Everything from allocated overhead, supplies, attributed salaries, and other expenses may be included. The actual values, supplemental data and expenditure attribution 540 data may be aggregated at performance comparison 545 and provided to the performance matrix 400. This preprocessing and arrangement of data into the matrix format facilitates the grouping of patient data, making determination of groups less resource intensive.

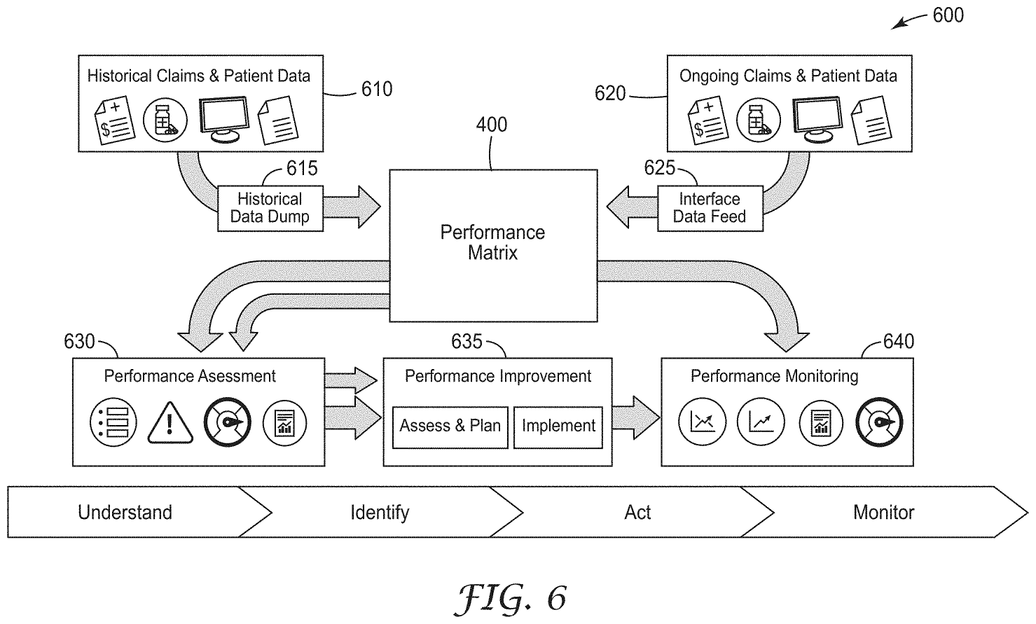

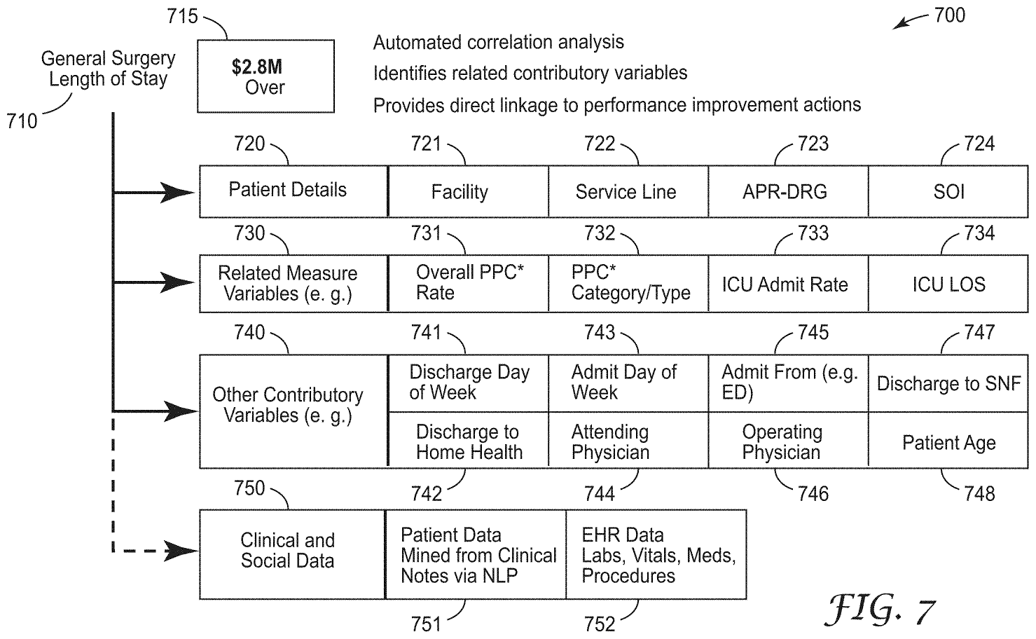

FIG. 6 is a block flow diagram illustration operation of the system 600 utilizing the performance matrix 400. Historical claims and patient data 610 is provided in a historical data dump represented at 615 to the performance matrix 400 to create and augment the matrix. Ongoing claims and patient data 620 is provided as an interface data feed 625 to provide current information into the performance matrix. Using the performance matrix 400, the system performs performance assessment 630, allowing performance improvement 635 via assessing, planning, and implementation. Monitoring of the performance is also performed over time at 640. Once improvements are implemented the performance matrix maybe updated to reflect a selected period since the improvement implementation such that the data derived from the performance matrix reflects data collected after implementation. The difference in performance shows the value obtained via the improvement implementation over a group of patients that is comparable to the original group on which performance was assessed.

The system 600 allows a user to understand performance opportunities by associated details correlated to performance opportunities, as well as identifying potential improvements based on such details. Users may then act to implement one or more improvements and then monitor the performance to see how the improvement increased or decreased efficiencies/performance.

FIG. 7 is graphic representation of a performance improvement identification 700 by the system. In one example, a primary performance measure is shown as length of stay for general surgery indicated at 710. A calculated cost savings opportunity is illustrated at 715 as $2.8M. Patient details are shown at 720 and include facility 721, service line 722, APR-DRG 723, and SOI 724. Related measure variables 730 includes for example, overall PPC (potentially preventable complication) 731, PPC category/type 732, ICU admit rate 733 and ICO LOS 734. Other contributory variables 740 include discharge day of week 741, discharge to home health 742, admit day of week 743, attending physician 744, admit from (e.g. ED) 745, operating physician 746, discharge to SNF 747, and patient age 748.

Finally, clinical and social data 750 may be included in further embodiments and includes patient data mined from clinical notes via natural language processing 751 and HER data such as labs, vitals, meds, procedures 752.

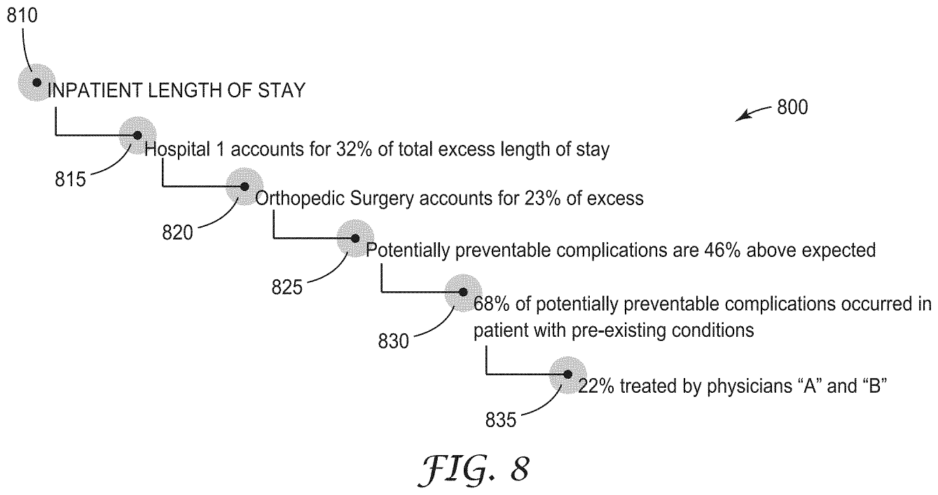

FIG. 8 is a graphical representation 800 of relevant causes for the primary performance measure of inpatient length of stay at 810. The causes are represented indented in descending order of relevance. At 815 it is noted that hospital 1 accounts for 32% of total excess length of stay encounters. 820 indicated orthopedic surgery accounts for 23% of excess stays. 825 indicates that potentially preventable complications are 46% above expected. 830 indicates that 68% of potentially preventable complications occurred in patients with pre-existing conditions, and 835 indicates that 22% of excess length of stay encounters resulted from treatment by physicians “A” and “B”.

Each of these causes point to areas to investigate and at a minimum question, leading to identification of particular actions that may be implemented to reduce the percentages associated with the cause. For instance, what is hospital 1 doing that results in 32% of their patients having longer than average stays for this type of procedure? How could the complications be prevented with respect to cause 825? What are the preexisting conditions that results in preventable complications at cause 830 and how can we better prevent them? As can be seen, the data provided by the system is simple to understand and clearly points to areas to investigate to improve efficiency as well as outcomes and saving expense.

Note that the causes are indented, with each cause pointing to more specific actionable issues the further such causes are indented, while those less indented identify broader issues that may be more difficult to address.

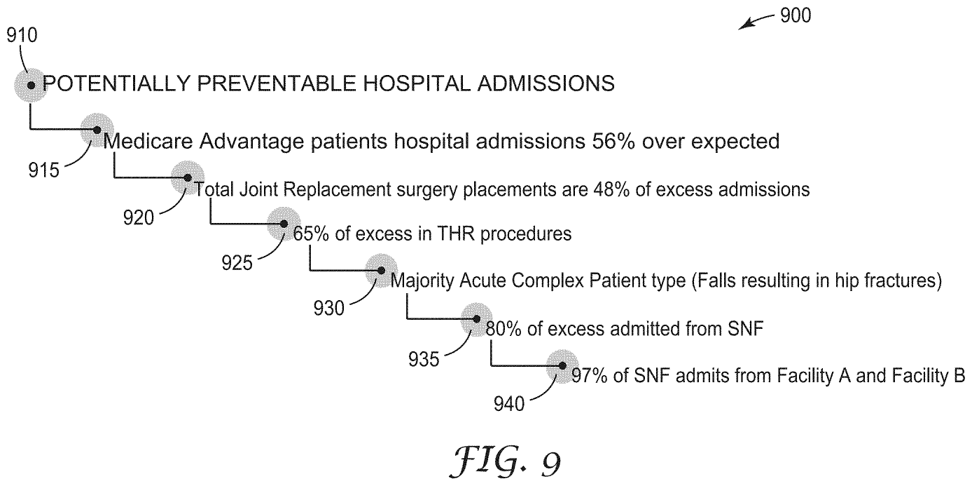

FIG. 9 is a graphical representation 900 of relevant causes for the primary performance measure of potentially preventable hospital admissions 910. Corresponding causes are shown at 915, 920, 925, 930, 935, and 940 with corresponding benchmark deviations expressed as percentages or particular patient types. Again, the causes directly suggest areas for investigation and identification of improvements in process.

In various embodiments, the system defines the patient group, by identifying the health care facility associated with the poor performing patient group, service lines within the facility associated with the poor performing patient group, diagnosis category of the patient group, and severity of illness level of the patients.

The system then evaluates the performance of the patients in the group on other related performance measures (secondary measures) that may be related to or causing the poor performance on the primary measure.

Then the system identifies other details about the patient group that may be correlated to the patients performing poorly on the primary performance measure. The associated details may include:
Attending physician
Operating physician
Patient age
Patient zip code
Nursing unit
Admit day of the week
Discharge day of the week
Admit from location
Discharge to location

The outcome of the PIES system is a focused area of potential improvement that is quantified and includes the associated details related to this opportunity that enable a plan to be quickly put together in order to address and solve the underlying issue behind the poor performance.

Example Application Scenario:

In evaluating the performance of a hospital, the system finds that there is a group of patients that have inpatient length of stay (LOS) that is longer than the expected standard benchmark for that particular patient group, measured in bed days. The system identifies that this is occurring at Hospital A, in the Orthopedic Service Line, with patient diagnosis group 58, and specifically with high severity of illness (SOI) group of patients in diagnosis group 58. The system also calculated that this poor performance is worth $Z if the length of stay for this patient group were to be improved up to the benchmark expected level.

The system then evaluates this patient group for potentially preventable complications that may have occurred during their hospital stay and compares the rates of complications against a benchmark rate of expected complications. The system finds that this patient group has a rate of complication type 16 that is higher than expected as compared to a benchmark rate of occurrence for this specific patient group. In this scenario, patients getting a complication while in the hospital, will require longer stays to deal with the effects of the complication.

The system then identifies which physicians (attending and operating/surgeon) were involved in this patient group and finds that physicians Dr. Attending X and Dr. Surgeon Y were involved with a large percentage of the involved patients. The system also finds that a large percentage of these patients were admitted on a weekend day and were discharged to skilled nursing facilities.

The result is that the system found:
Performance Improvement Opportunity for Length of Stay worth $Z
The Opportunity is at Facility A, in the Orthopedic Service Line, with Patient Group 58, with the High SOI patients
The secondary measure contributors to this Opportunity are potentially preventable complication type 16
Physicians Dr. Attending X and Dr. Surgeon Y are involved
Weekend admissions and discharges to skilled nursing facilities are also related to this opportunity

Thus, a performance improvement project would focus on this particular patient type at Facility A and evaluate the involved physicians, along with the processes involved in weekend admissions and discharges to skilled nursing facilities that may be related to why this patient group has a high rate of complication type 16, which thus is contributing to the excess length of stay for this patient group.

While prior performance identification systems allowed users to look at canned reports and generate queries to attempt to identify areas for improvements, such systems were usually based on data organized by facility. No patient groups were identified, resulting in results that were based on data that may be skewed based on different characteristics of patients. While tedious queries may be generated with significant human effort, finding areas to improve was more of a hit or miss endeavor.

The present system provides for one or more of comprehensive assessment of improvement areas by utilizing grouping of patients and iterative processes to both identify areas of improvement, quantify the potential savings for such improvement, and provide data representative of causes for inefficient operation, pointing to actions that can be taken to realize cost savings.

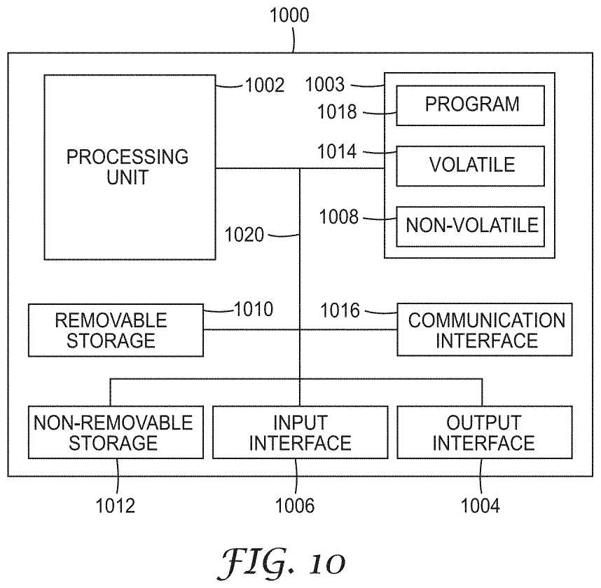

FIG. 10 is a block schematic diagram of a computer system 1000 to implement a system for identifying and quantifying areas for performance improvement and for performing methods and algorithms according to example embodiments. All components need not be used in various embodiments.

One example computing device in the form of a computer 1000 may include a processing unit 1002, memory 1003, removable storage 1010, and non-removable storage 1012. Although the example computing device is illustrated and described as computer 1000, the computing device may be in different forms in different embodiments. For example, the computing device may instead be a smartphone, a tablet, smartwatch, smart storage device (SSD), or other computing device including the same or similar elements as illustrated and described with regard to FIG. 10. Devices, such as smartphones, tablets, and smartwatches, are generally collectively referred to as mobile devices or user equipment.

Although the various data storage elements are illustrated as part of the computer 1000, the storage may also or alternatively include cloud-based storage accessible via a network, such as the Internet or server-based storage. Note also that an SSD may include a processor on which the parser may be run, allowing transfer of parsed, filtered data through I/O channels between the SSD and main memory.

Memory 1003 may include volatile memory 1014 and non-volatile memory 1008. Computer 1000 may include—or have access to a computing environment that includes—a variety of computer-readable media, such as volatile memory 1014 and non-volatile memory 1008, removable storage 1010 and non-removable storage 1012. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 1000 may include or have access to a computing environment that includes input interface 1006, output interface 1004, and a communication interface 1016. Output interface 1004 may include a display device, such as a touchscreen, that also may serve as an input device. The input interface 1006 may include one or more of a touchscreen, touchpad, mouse, keyboard, camera, one or more device-specific buttons, one or more sensors integrated within or coupled via wired or wireless data connections to the computer 1000, and other input devices. The computer may operate in a networked environment using a communication connection to connect to one or more remote computers, such as database servers. The remote computer may include a personal computer (PC), server, router, network PC, a peer device or other common data flow network switch, or the like. The communication connection may include a Local Area Network (LAN), a Wide Area Network (WAN), cellular, Wi-Fi, Bluetooth, or other networks. According to one embodiment, the various components of computer 1000 are connected with a system bus 1020.

Computer-readable instructions stored on a computer-readable medium are executable by the processing unit 1002 of the computer 1000, such as a program 1018. The program 1018 in some embodiments comprises software to implement one or more methods performed by the system, as well as generation of the performance matrix and providing actionable information for identifying performance improvements. A hard drive, CD-ROM, and RAM are some examples of articles including a non-transitory computer-readable medium such as a storage device. The terms computer-readable medium and storage device do not include carrier waves to the extent carrier waves are deemed too transitory. Storage can also include networked storage, such as a storage area network (SAN). Computer program 1018 along with the workspace manager 1022 may be used to cause processing unit 1002 to perform one or more methods or algorithms described herein.

### EXAMPLES

1. A computer implemented method includes obtaining access to a database containing patient healthcare data that includes identifications of health care facility, service lines, diagnosis category, severity of illness level, and multiple performance measures, analyzing, via the computer, the healthcare data to identify sets of patients sharing one or more of the identifications, analyzing, via the computer, the identified sets of patients to identify a group from one of the identified sets, the group including patients having a primary performance measure worse than a performance benchmark associated with the diagnosis category and severity of illness level of the one of the identified sets, analyzing, via the computer, secondary performance measures of the identified group to identify secondary performance measures correlated to the, primary performance measure being worse than the performance benchmark, and analyzing, via the computer, the healthcare data to identify associated details of patient care in the group corresponding to the primary performance measure being worse than the performance benchmark.

2. The method of example 1 wherein the analyzing the healthcare data to identify the associated details is performed as a function of the identified secondary performance measures.

3. The method of any of examples 1-2 wherein analyzing the identified sets of patients to identify a group comprises iteratively creating multiple sets as a function of at least one or more of health care facility, service lines, diagnosis category, and severity of illness.

4. The method of example 3 wherein analyzing the identified sets of patients to identify a group from one of the identified sets includes determining performance of each of the multiple sets, comparing the determined performance of each of the multiple sets to corresponding performance benchmarks to determine a difference for each set, and generating a list of sets based on the differences.

5. The method of example 4 and further comprising selecting the set with the largest difference as the identified group.

6. The method of any of examples 1-5 wherein the associated details include one or more of attending physician, operating physician, patient age, patient zip code, nursing unit, admit day of the week, discharge day of the week, admit from location, and discharge to location.

7. The method of example 6 wherein the associated details are ranked according to frequency of occurrence in the group to identify a focused area of potential improvement that is quantified by the associated difference.

8. The method of any of examples 1-7 wherein the primary performance measure comprises length of stay.

9. The method of example 8 and further comprising determining a cost associated with an average difference between the length of stay and a benchmark length of stay, wherein the group is selected as the group having the highest cost.

10. The method of any of examples 1-9 wherein performance measures include two or more of length of stay, readmissions, complications, pharmacy, laboratory, emergency room visits, radiology, and mortality.

11. A machine-readable storage device has instructions for execution by a processor of a machine to cause the processor to perform operations to perform a method of grouping patients to identify areas for improvement of healthcare delivery. The operations include obtaining access to a database containing patient healthcare data that includes identifications of health care facility, service lines, diagnosis category, severity of illness level, and multiple performance measures, analyzing, via the computer, the healthcare data to identify sets of patients sharing one or more of the identifications, analyzing, via the computer, the identified sets of patients to identify a group from one of the identified sets, the group including patients having a primary performance measure worse than a performance benchmark associated with the diagnosis category and severity of illness level of the one of the identified sets, analyzing, via the computer, secondary performance measures of the identified group to identify secondary performance measures correlated to the primary performance measure being worse than the performance benchmark, and analyzing, via the computer, the healthcare data to identify associated details of patient care in the group corresponding to the primary performance measure being worse than the performance benchmark.

12. The device of method 11 wherein the analyzing the healthcare data to identify the associated details is performed as a function of the identified secondary performance measures.

13. The device of any of examples 11-12 wherein analyzing the identified sets of patients to identify a group comprises iteratively creating multiple sets as a function of at least one or more of health care facility, service lines, diagnosis category, and severity of illness.

14. The device of method 13 wherein analyzing the identified sets of patients to identify a group from one of the identified sets includes determining performance of each of the multiple sets, comparing the determined performance of each of the multiple sets to corresponding performance benchmarks to determine a difference for each set, and generating a list of sets based on the differences.

15. The device of method 14 and further comprising selecting the set with the largest difference as the identified group.

16. The device of any of examples 11-15 wherein the associated details include one or more of attending physician, operating physician, patient age, patient zip code, nursing unit, admit day of the week, discharge day of the week, admit from location, and discharge to location.

17. The device of method 16 wherein the associated details are ranked according to frequency of occurrence in the group to identify a focused area of potential improvement that is quantified by the associated difference.

18. A device comprising a processor and a memory device coupled to the processor and having a program stored thereon for execution by the processor to perform operations. The operations include obtaining access to a database containing patient healthcare data that includes identifications of health care facility, service lines, diagnosis category, severity of illness level, and multiple performance measures, analyzing, via the computer, the healthcare data to identify sets of patients sharing one or more of the identifications, analyzing, via the computer, the identified sets of patients to identify a group from one of the identified sets, the group including patients having a primary performance measure worse than a performance benchmark associated with the diagnosis category and severity of illness level of the one of the identified sets, analyzing, via the computer, secondary performance measures of the identified group to identify secondary performance measures correlated to the primary performance measure being worse than the performance benchmark, and analyzing, via the computer, the healthcare data to identify associated details of patient care in the group corresponding to the primary performance measure being worse than the performance benchmark.

19. The device of method 18 wherein the analyzing the healthcare data to identify the associated details is performed as a function of the identified secondary performance measures, and wherein analyzing the identified sets of patients to identify a group comprises iteratively creating multiple sets as a function of at least one or more of health care facility, service lines, diagnosis category, and severity of illness.

20. The device of method 19 wherein analyzing the identified sets of patients to identify a group from one of the identified sets includes determining performance of each of the multiple sets, comparing the determined performance of each of the multiple sets to corresponding performance benchmarks to determine a difference for each set, and generating a list of sets based on the differences.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A computer implemented method comprising:
obtaining access to a database containing patient healthcare data that includes identifications of health care facility, service lines, diagnosis category, severity of illness level, and multiple performance measures;
analyzing, via the computer, the healthcare data to identify sets of patients sharing one or more of the identifications;
analyzing, via the computer, the identified sets of patients to identify a group from one of the identified sets, the group including patients having a primary performance measure worse than a performance benchmark associated with the diagnosis category and severity of illness level of the one of the identified sets;
analyzing, via the computer, secondary performance measures of the identified group to identify secondary performance measures correlated to the primary performance measure being worse than the performance benchmark; and
analyzing, via the computer, the healthcare data to identify associated details of patient care in the group corresponding to the primary performance measure being worse than the performance benchmark.

2. The method of claim 1 wherein the analyzing the healthcare data to identify the associated details is performed as a function of the identified secondary performance measures.

3. The method of claim 1 wherein analyzing the identified sets of patients to identify a group comprises iteratively creating multiple sets as a function of at least one or more of health care facility, service lines, diagnosis category, and severity of illness.

4. The method of claim 3 wherein analyzing the identified sets of patients to identify a group from one of the identified sets includes:
determining performance of each of the multiple sets;
comparing the determined performance of each of the multiple sets to corresponding performance benchmarks to determine a difference for each set; and
generating a list of sets based on the differences.

5. The method of claim 4 and further comprising selecting the set with the largest difference as the identified group.

6. The method of claim 1 wherein the associated details include one or more of attending physician, operating physician, patient age, patient zip code, nursing unit, admit day of the week, discharge day of the week, admit from location, and discharge to location.

7. The method of claim 6 wherein the associated details are ranked according to frequency of occurrence in the group to identify a focused area of potential improvement that is quantified by the associated difference.

8. The method of claim 1 wherein the primary performance measure comprises length of stay.

9. The method of claim 8 and further comprising determining a cost associated with an average difference between the length of stay and a benchmark length of stay, wherein the group is selected as the group having the highest cost.

10. The method of claim 1 wherein performance measures include two or more of length of stay, readmissions, complications, pharmacy, laboratory, emergency room visits, radiology, and mortality.

11. A machine-readable storage device having instructions for execution by a processor of a machine to cause the processor to perform operations to perform a method of grouping patients to identify areas for improvement of healthcare delivery, the operations comprising:
obtaining access to a database containing patient healthcare data that includes identifications of health care facility, service lines, diagnosis category, severity of illness level, and multiple performance measures;
analyzing, via the computer, the healthcare data to identify sets of patients sharing one or more of the identifications;
analyzing, via the computer, the identified sets of patients to identify a group from one of the identified sets, the group including patients having a primary performance measure worse than a performance benchmark associated with the diagnosis category and severity of illness level of the one of the identified sets;
analyzing, via the computer, secondary performance measures of the identified group to identify secondary performance measures correlated to the primary performance measure being worse than the performance benchmark; and
analyzing, via the computer, the healthcare data to identify associated details of patient care in the group corresponding to the primary performance measure being worse than the performance benchmark.

12. The device of claim 11 wherein the analyzing the healthcare data to identify the associated details is performed as a function of the identified secondary performance measures.

13. The device of claim 11 wherein analyzing the identified sets of patients to identify a group comprises iteratively creating multiple sets as a function of at least one or more of health care facility, service lines, diagnosis category, and severity of illness.

14. The device of claim 13 wherein analyzing the identified sets of patients to identify a group from one of the identified sets includes:
determining performance of each of the multiple sets;
comparing the determined performance of each of the multiple sets to corresponding performance benchmarks to determine a difference for each set; and
generating a list of sets based on the differences.

15. The device of claim 14 and further comprising selecting the set with the largest difference as the identified group.

16. The device of claim 11 wherein the associated details include one or more of attending physician, operating physician, patient age, patient zip code, nursing unit, admit day of the week, discharge day of the week, admit from location, and discharge to location.

17. The device of claim 16 wherein the associated details are ranked according to frequency of occurrence in the group to identify a focused area of potential improvement that is quantified by the associated difference.

18. A device comprising:
a processor; and
a memory device coupled to the processor and having a program stored thereon for execution by the processor to perform operations comprising:
obtaining access to a database containing patient healthcare data that includes identifications of health care facility, service lines, diagnosis category, severity of illness level, and multiple performance measures;
analyzing, via the computer, the healthcare data to identify sets of patients sharing one or more of the identifications;
analyzing, via the computer, the identified sets of patients to identify a group from one of the identified sets, the group including patients having a primary performance measure worse than a performance benchmark associated with the diagnosis category and severity of illness level of the one of the identified sets;
analyzing, via the computer, secondary performance measures of the identified group to identify secondary performance measures correlated to the primary performance measure being worse than the performance benchmark; and
analyzing, via the computer, the healthcare data to identify associated details of patient care in the group corresponding to the primary performance measure being worse than the performance benchmark.

19. The device of claim 18 wherein the analyzing the healthcare data to identify the associated details is performed as a function of the identified secondary performance measures, and wherein analyzing the identified sets of patients to identify a group comprises iteratively creating multiple sets as a function of at least one or more of health care facility, service lines, diagnosis category, and severity of illness.

20. The device of claim 19 wherein analyzing the identified sets of patients to identify a group from one of the identified sets includes:
determining performance of each of the multiple sets;
comparing the determined performance of each of the multiple sets to corresponding performance benchmarks to determine a difference for each set; and
generating a list of sets based on the differences.
